(19) United States
(12) Patent Application Publication
(10) Pub. No.: US 2018/0160864 A1
(43) Pub. Date: Jun. 14, 2018

(54) SYMMETRICAL SKIN SCRAPER

(71) Applicant: Dirk Wehlmann, Wiesbaden (DE)

(72) Inventor: Dirk Wehlmann, Wiesbaden (DE)

(21) Appl. No.: 15/739,284

(22) PCT Filed: Jun. 22, 2016
(86) PCT No.: PCT/EP2016/064424

§ 371 Date: Dec. 22, 2017

(30) Foreign Application Priority Data
Jun. 25, 2015 (DE) 202015004504.9

(51) Int. Cl. A47K 7/02; A61B 17/54; A61B 10/00; A61B 10/02

(52) U.S. Cl. CPC A47K 7/028; A61B 17/54; A61B 2017/320008; A61B 10/02; A61B 10/0064

(57) ABSTRACT

The invention relates to a skin scraper in the form of a blade, characterized in that said blade has two long sides a and b and two smaller sides c and d, the sides a and be having the same shape.

The invention relates to an advantageous further development of a skin scraper of the kind described in German patent application no. 10 2014 006 742.4. In that application, a skin scraper is disclosed which comprises a blade which has two long sides a and b, whereby one side, the side a, has a concave shape, and the other side b is different in its shape from side a and is e.g. it is a straight line.

The skin scrapers described therein are especially suited for the treatment, massage, cleaning and purification of the skin, especially for use during a stay in a sauna, but also for the stripping or collecting of sweat water, and skin residues which can thereafter be subjected to analysis or examination.

The present invention provides an advantageous further development of a skin scraper of this kind.

In U.S. Pat. No. 1,651,085 a skin cleaner is described which is suitable for the superficial separation of cosmetics such as rouge and cold cream from the skin, in particular from the skin of the face, and which comprises a blade which has a handle, preferentially on both sides. The blade consists of a flexible material e.g. a fibrous product and is bent 180° in order to separate cream residues from facial skin so that one can scrape cosmetic residues off the exterior of the skin.

This device, however, is not suitable for massaging or the provision of a deep cleaning of the skin, e.g. when used in the sauna.

Korean patent application 1020130013087 describes a device which has the shape of an arrow-head, has a variety of bulges and is useful for the massage of thumbs, arms and Achilles tendons.

The device has no cleaning function and does not allow collecting impurities.

In U.S. Pat. No. 4,438,767 a circular skin scraper is described which on account of its exterior shape is not comparable with a skin scraper according to the invention.

WO 2007/137322 A1 describes a skin treatment tool which consists of a sheath and a blade arranged so that the blade has one side in the sheath and the other free side is convexly curved and the device is useful for massage and removing dead skin cells from the skin and does provide deep cleaning or collecting possibilities

The shapes of both sides are, however, not the same.

Although a large number of skin scrapers are known, there is still a demand for improved skin scrapers, in particular, for those which allow a deep cleaning of the skin and which are, on account of their shape, more comfortable to handle.

An object of the invention is to provide a skin scraper which is more easily and more comfortably handled during utilization, which is easy to manufacture and which, in particular, may be used comfortably in a sauna, for many purposes and can be used effectively by oneself or by service personnel.

This object is solved by a skin scraper which comprises a blade with two sides a and b and two smaller sides c and d, whereby the sides a and b have the same shape.

The sides a and b preferentially have a linear, convex or concave shape.

The skin scraper is preferentially connected at one of its smaller sides to a handle e.

The blade can be flat, but preferentially has a concave shape. Preferentially, there is an additional groove parallel to the sides a and b.

The groove is preferentially equidistant from the sides a and b.

In an advantageous embodiment of the invention one of the smaller sides has one or two bulges. These bulges can have different radii of curvature.

The edges of the sides a, b and c of the blade are advantageously curved or tapered.

It is advantageous to provide finger protection at the location of the transition between the blade and the handle, which serves for the protection and support of the thumb and a finger. When the blade is e.g. concavely curved, this curvature is preferentially over the entire length of the blade.

The skin scraper according to the invention is especially suited for massage, treating, cleaning and purification of the skin, especially during a stay in a sauna.

With the help of the skin scraper liquid and solid skin discharges may be collected via the groove and transported for analysis. Treatment with the skin scraper fosters skin respiration and the purification and detoxification of the skin.

The collected matter may be collected for analysis or examination purposes and subjected to a medical or diagnostic examination.

It is particularly advantageous that the skin scraper according to the invention may be used quite comfortably when one treats, e.g. his or her leg in a lying position. Thus one can, without changing the position of the skin scraper in the hand, massage the leg from above as well from below to remove sweat. Also, e.g. during darkness, one need not look at the location of the blade with which one wants to treat the skin because either side of the blade may be used for the same function.

The use of the skin scraper is very simple. One strokes the skin surface with a certain pressure, e.g. at an angle of under 20°. It is possible, by varying the applied pressure to move from starting with a simple surface effect such as stripping humidity or loose particles from the skin, to deep cleaning effect. This takes place when one strokes or presses the sebaceous glands or sweat pores in a sauna of the previously warmed and humidified skin surface with the specially formed edge of the blade.

The collection of the liquid which is contains a high level of impurities, and separated particles, which are gathered during the treating of the skin in the hollow part of the scraper blade, is extremely simple. From there one can easily transfer the collected liquids or dispersions into a collecting vessel and subject them further to analysis or examination.

As a result of this deep cleaning of the pores, the visitor to the sauna realizes considerable relief and an augmentation of his well-being. Further, skin respiration and transpiration is facilitated and fostered and thus the effect of a traditional sauna visit is improved.

The length of the sides a and b can vary within large limits. Thus e.g. lengths of 5 to 30 cm are suitable. The breadth can vary within large limits as well, e.g. 2 to 5 cm. Suitable materials for the blade are non-rusting metals or alloys, non-rusting steel or plastics such as plexiglass, horn and ceramics. The production of a scraper according to the invention is illustrated in the following examples.

### EXAMPLE 1

A shaped body is stamped from a 1 mm thick high grade steel sheet having a length of 25 cm and a breadth of 5 cm. A concave blade is formed. At a small side a handle is fixed with which the skin scraper can easily be moved by one hand.

### EXAMPLE 2

Using a scraper according to Example 1 the skin, which has been warmed and humidified in a sauna, is scraped off at an angle of 20° with a variable pressure on the leg. Thereby the sweat, which is enriched with impurities, is collected in the groove and transferred via an outlet to a separate vessel. Thereafter, the composition of the liquid is analyzed.

It especially advantageous that according to the invention the visitor of the sauna can handle the skin scraper in both the sitting and lying positions. Even during darkness the user need not look at the location which has to be moved over the skin for treatment because an equivalent treatment is possible from both sides of the device.

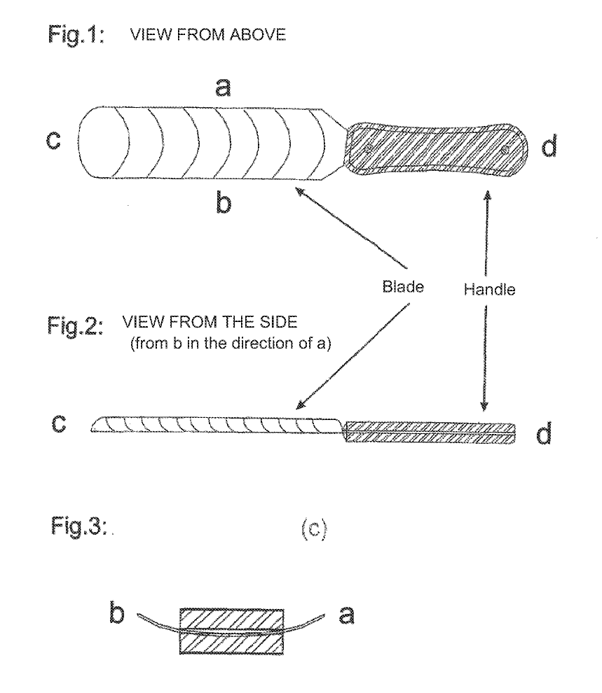

The invention is explained in greater detail with reference to FIGS. 1, 2, and 3.

FIG. 1 presents a blade according to the invention, where both long sides a and b have the same shape, i.e. the blade is concave shaped. At the right side there is a handle which fits well into the hand. From the side c the sweat which is collected in the concave groove can be poured out.

FIG. 2 shows a side view of the skin scraper.

FIG. 3 shows a front view of the skin scraper, i.e. from the direction of the small side c. In this view, one can distinctly see that the blade is concavely curved. The handle is indicated by the square object.

1) A skin scraper in a form of blade characterized in that the blade has two long sides a and b and two smaller sides c and d whereby the sides a and b have a same shape.

2) The skin scraper according to claim 1, characterized in that the long sides a and b have a linear or concave or convex shape.

3) The skin scraper according to claim 1, characterized in that one said smaller side is connected with a handle e.

4) The skin scraper according to claim 1, characterized in that the blade is flat.

5) The skin scraper according to claim 1, characterized in that the blade is concave.

6) The skin scraper according to claim 1, characterized in that the blade has a groove parallel to the sides a and b.

7) Skin scraper according to claim 6 characterized in that the groove is equidistant from the sides a and b.

8) The skin scraper according to claim 1, characterized in that one of the smaller sides has one or two bulges.

9) The skin scraper according to claim 8, characterized in that the bulges have different radii of curvature.

10) The skin scraper according to claim 1, characterized in that edges of the sides a and b and c of the blade are curved or tapered.

11) The skin scraper according to claim 1, characterized in that there is located at a transition between the blade and the handle, a finger protection f is for protecting and supporting a thumb and a finger.

12) The skin scraper according to claim 2, characterized in that one said smaller side is connected with a handle e.

13) The skin scraper according to claim 3, characterized in that the blade is flat.

14) The skin scraper according to claim 3, characterized in that the blade is concave.

15) The skin scraper according to claim 4, characterized in that the blade has a groove parallel to the sides a and b.

16) Skin scraper according to claim 15 characterized in that the groove is equidistant from the sides a and b.

17) The skin scraper according to claim 4, characterized in that one of the smaller sides has one or two bulges.

18) The skin scraper according to claim 17, characterized in that the bulges have different radii of curvature.

19) The skin scraper according to claim 5, characterized in that the blade has a groove parallel to the sides a and b and the groove is equidistant from the sides a and b.

20) The skin scraper according to claim 4, characterized in that one of the smaller sides has one or two bulges and the bulges have different radii of curvature.
